(19) United States
(12) Patent Application Publication
(10) Pub. No.: US 2025/0289606 A1
(43) Pub. Date: Sep. 18, 2025

(54) FUNCTIONAL ELEMENT, BETA CONTAINER SYSTEM, TRANSFER SYSTEM AND BARRIER SYSTEM

(71) Applicant: Groninger & Co. GmbH, Crailsheim (DE)

(72) Inventor: Armin MERZ, Ellwangen (DE)

(21) Appl. No.: 19/220,634

(22) Filed: May 28, 2025

Related U.S. Application Data
(63) Continuation of application No. PCT/EP2023/081767, filed on Nov. 14, 2023.

(30) Foreign Application Priority Data
Nov. 30, 2022 (DE) 10 2022 131 808.7

(51) Int. Cl. B65B 55/02; B65B 7/28

(52) U.S. Cl. CPC B65B 55/027; B65B 7/2807

(57) ABSTRACT

A functional element for a beta container system, wherein the functional element has a functional element opening. The functional element opening is smaller than a beta port opening of a beta port of a beta container of the beta container system. The functional element can be arranged on the beta container in a defined arrangement, the functional element is arranged at least partially in the beta port opening in the defined arrangement, and a bag of the beta container is arranged in the defined arrangement between the beta port and the functional element. A beta container system for a transfer system, a transfer system, and a barrier system, in particular an isolator system, are also disclosed.

### CROSS-REFERENCE TO RELATED APPLICATIONS

This is a Continuation application of International patent application PCT/EP2023/081767, filed Nov. 14, 2023, which claims the priority of German patent application DE 10 2022 131 808.7 filed Nov. 30, 2022. Both application PCT/EP2023/081767 and DE 10 2022 131 808.7 are herewith incorporated by reference in their entireties.

### FIELD

The present invention generally relates to a functional element for a beta container system, to a beta container system for a transfer system wherein the beta container system comprises a beta container and such a functional element, to a transfer system with such a beta container, and to a barrier system, in particular an isolator system, with such a transfer system.

### BACKGROUND

A barrier system is a system that provides a physical and aerodynamic barrier, e.g., by means of positive air pressure, between an external environment, for example an external cleanroom environment, and a work process. Various barrier systems are known in the prior art. A barrier system can, for example, be an isolator or a barrier with restricted access, a so-called RABS (restricted area barrier system). The RABS can be an open RABS or a closed RABS.

The present disclosure is primarily concerned with aseptic isolators as barrier systems. However, the present disclosure can also be used in other barrier systems.

The term “isolator” generally refers to a container that is hermetically and gas-tightly sealed from the surrounding work space. A defined atmosphere can be created within an isolator for processing sensitive or dangerous products.

In this context, isolators are typically used in biopharmaceutical process engineering, for example as part of a filling system with multiple process and processing stations, to create a highly clean or sterile, i.e., germ-free, environment.

In such filling systems, for example, containers, in particular vials, cartridges, bottles, syringes and/or the like, can be filled with a product, preferably a pharmaceutical or cosmetic product, in particular a liquid or a powder, and then closed with a closure element, for example a stopper or a closure cap, in particular a crimp cap.

Pharmaceutical filling systems are usually located in a low-germ environment. In particular, a germ-free environment must be present in the filling area within the isolator. This state is monitored by placing culture medium carriers, also called germ collectors, at the critical points. Culture medium carriers can, for example, be Petri dishes that contain a culture medium. If a germ is placed onto the culture medium, the germ will grow during subsequent incubation, which allows contamination to be retrospectively detected. Such monitoring of a germ-free environment is called “germ monitoring” or “microbiological monitoring.”

Transfer systems can be used to transfer objects, such as closure elements or culture medium carriers, into or out of the isolator. A transfer system can have a transfer lock that can be coupled to the isolator from the outside. Objects can be transferred into or out of the isolator via the transfer lock. A transfer system can, for example, be designed as a port system, in particular as an alpha-beta port system. Such transfer systems have an alpha port and a beta container. The beta container serves as a transfer lock. The beta container has a beta port. The alpha port is preferably arranged on an isolator wall of the isolator surrounding an interior of the isolator. The alpha port and the beta port can be coupled together to connect the interior of the isolator to an internal volume of the beta container. In the coupled state, objects can be brought from the beta container into the isolator, or objects can be inserted from the isolator into the beta container.

Feed devices can be used to feed closure elements provided in a beta container. A feed device can be arranged within the isolator and can be arranged from the inside at the alpha port. The feed device can, for example, comprise a chute or a tube. The closure elements can then be inserted or fed from the beta container into the isolator via the feed devices.

For example, the publication DE 10 2021 101 384 B3 shows a system for transporting sterile pourable closure elements from an environment of an isolator into an interior of the isolator, comprising a container for storing a supply quantity of closure elements in the environment of the isolator, an isolator opening and a collecting device for collecting the closure elements and for providing the closure elements in the interior of the isolator, with a dosing apparatus for controlling a desired quantity of closure elements to be transported from the container through the isolator opening and into the collecting device.

Furthermore, publication EP 3 581 339 B1 shows a transfer system for a sealed housing, wherein the sealed housing defines a first closed volume and has at least one sealed connection apparatus designed to connect the first closed volume to a second closed volume, wherein the transfer system is designed to be arranged in the housing, wherein the transfer system comprises at least one arm designed to be rotatably mounted on a wall of the sealed housing via a first pivot joint having a first axis of rotation, wherein the transfer system comprises a chute, wherein the chute has a docking edge and a pouring edge, wherein the docking edge is designed to cooperate with the sealed connection apparatus, wherein the transfer system comprises a second pivot joint between the arm and the chute, wherein the second pivot joint has a second axis of rotation.

A port system can also be used to feed or remove culture medium carriers into or out of an isolator. The culture medium carriers can be provided in particular in a beta container. For example, the isolator can be provided with a handling device that can remove the culture medium carriers from the beta container or return it into it. Alternatively, culture medium carriers slides can also be removed from or returned to the beta container manually using a gloved hand.

For example, the publication DE 10 2020 102 758 B4 shows a method for automated germ monitoring in an isolator, wherein the isolator has a transfer lock, the method comprising the following steps: first provision of at least one culture medium carrier holder at a respective first position within the isolator; second provision of at least one culture medium carrier within the transfer lock; first robot-assisted transfer of a respective individual culture medium carrier of the at least one culture medium carrier from the transfer lock to a free culture medium carrier holder of the at least one culture medium carrier holder; and first robot-assisted arranging of the transferred culture medium carrier in the free culture medium carrier holder. Furthermore, this publication shows a system for automated germ monitoring in an isolator and a computer program.

However, the known transfer systems still leave room for improvement, in particular with regard to structural design, the transfer of objects and operational safety.

### SUMMARY

Against this background, it is an object of the present application to improve the structural design of a transfer system and a barrier system. Furthermore, it is an object of the present application to improve the transfer of objects in a transfer system and a barrier system. Furthermore, it is an object of the present application to improve the operational safety of a transfer system and a barrier system.

According to a first aspect, a functional element for a beta container system is provided, wherein the functional element has a functional element opening, wherein the functional element opening is smaller than a beta port opening of a beta port of a beta container of the beta container system, wherein the functional element can be arranged on the beta container in a defined arrangement, wherein the functional element is arranged at least partially in the beta port opening in the defined arrangement, and wherein a bag of the beta container is arranged in the defined arrangement between the beta port and the functional element.

According to a second aspect, a beta container system for a transfer system is provided, wherein the beta container system comprises a beta container and the functional element according to the first aspect, wherein the beta container has an internal volume, a bag which encloses the internal volume, and a beta port with a beta port opening, wherein the beta port can be coupled to an alpha port of the transfer system.

According to a third aspect, a transfer system is provided, the transfer system comprising an alpha port and the beta container according to the third aspect.

According to a fourth aspect, there is provided a barrier system, wherein the barrier system comprises the transfer system according to the fourth aspect. The barrier system is, in particular, an isolator system with an isolator.

The isolator can have an interior. The isolator can have an isolator wall that encloses or surrounds the interior. The isolator wall separates the interior from an outer environment surrounding the isolator. The isolator is preferably an aseptic isolator. An aseptic isolator has a highly clean or sterile, i.e., germ-free, environment in the interior.

The isolator can preferably be part of a filling system with several process and processing stations. The filling system can, for example, be a system for filling and closing containers with a pharmaceutical or cosmetic substance. The system can in particular comprise a filling station and at least one closing station.

The transfer system is used to transfer objects, in particular closure elements or culture medium carriers, into or out of the isolator. The transfer system has the alpha port and the beta container.

The alpha port can be arranged on the isolator wall of the isolator. The alpha port has an alpha port opening. The alpha port opening forms a passage through the isolator wall. In other words, the isolator is accessible from the outside through the alpha port opening. The alpha port opening can have any shape. For example, the alpha port opening can be circular, elliptical, or rectangular. In a preferred refinement, the alpha port opening is circular.

The alpha port can also have an alpha port main body. The alpha port main body can preferably be ring-shaped. In particular, the alpha port main body can extend through the isolator wall. The alpha port main body has an inner side and an outer side. The inner side faces the interior. The outer side faces the outer environment. The outer side is in particular a side of the alpha port facing the beta port. The alpha port main body can include the alpha port opening. In particular, the alpha port opening can be formed by a recess in the alpha port main body that extends from the inner side to the outer side of the alpha port main body. In particular, the alpha port main body surrounds the alpha port opening. The alpha port can have an alpha port flange on the outer side. In particular, the alpha port main body can form the alpha port flange on the outer side. The alpha port flange surrounds the alpha port opening.

The alpha port can also have a door. The door is arranged at the alpha port opening of the alpha port. The door is used to open and close the alpha port opening. Preferably, the door is arranged to be movable, in particular pivotable, on the alpha port main body of the alpha port. The door can be moved to open or close the alpha port opening. In the closed state, the door tightly seals the alpha port opening. For example, the alpha port can have a drive device to move the door.

The beta container has an internal volume. Objects such as closure elements or culture medium carriers can be arranged in the internal volume. The beta container can have a bag. The bag surrounds or encloses the internal volume. The bag can be a flexible bag, in particular a sterile bag. The bag can be made of plastics material. The bag can have a bag wall. The bag wall can be flexible or resilient. In particular, the bag wall can be a plastics film.

The beta container has the beta port. The beta port can be arranged on the bag of the beta container. In particular, the beta port is connected to the bag. The beta port has a beta port opening. The beta port opening forms a passage through the bag wall to the internal volume. In other words, the internal volume is accessible from the outside through the beta port opening. The beta port opening can have any shape. For example, the beta port opening can be circular, elliptical, or rectangular. In a preferred refinement, the beta port opening is circular.

The beta port can also have a beta port main body. The beta port main body can preferably be ring-shaped. In particular, the beta port main body can extend into the internal volume. The beta port main body has an inner side and an outer side. The inner side faces the internal volume. The outer side faces the outer environment. The outer side is in particular a side of the beta port facing the alpha port. The beta port main body can include the beta port opening of the beta port. In particular, the beta port opening can be formed by a recess in the beta port main body extending from the inner side to the outer side of the beta port main body. In particular, the beta port main body surrounds the beta port opening. The beta port can have a beta port flange on the outer side. In particular, the beta port main body can form the beta port flange on the outer side. The flange surrounds the beta port opening.

The beta port can also have a cover element. The cover element can be arranged on or in the beta port opening of the beta port. The cover element is used to cover or close the beta port opening. The cover element can be detachably coupled to the beta port main body or the beta port flange. When the cover element is coupled to the beta port main body or the beta port flange, the cover element tightly closes the beta port opening and completely covers it.

The alpha port can be coupled to the beta port. In a coupled state, the internal volume of the beta container is connected to the interior of the isolator. In the coupled state, the alpha port and the beta port are coupled in such a way that the internal volume and the interior are isolated from the outer environment. In particular, a seal can be arranged between the alpha port and the beta port. In the coupled state, the beta port flange can be arranged on the alpha port flange. In particular, the seal can be arranged between the alpha port flange and the beta port flange.

The beta port opening and the alpha port opening are preferably the same size. In a preferred refinement, the beta port opening and the alpha port opening are circular. In particular, the beta port opening and the alpha port opening can have the same diameter.

For coupling purposes, the alpha port and the beta port may, for example, have corresponding coupling elements, wherein one or more coupling elements of the alpha port can be coupled to one or more coupling elements of the beta port. The coupling elements can be arranged on the alpha port main body and the beta port main body or on the beta port flange and the alpha port flange.

The door and the cover element can also be coupled. Only when the door and the cover element are coupled can the door and the cover element be moved together. In particular, they can be moved in such a way that the alpha port opening and the beta port opening can be opened and closed together. In particular, the door and the cover element can couple together when the alpha port and the beta port are coupled together. This allows the alpha port opening and the beta port opening to be opened and closed together when the alpha port and the beta port are coupled together.

For coupling purposes, the door and the cover element can, for example, have corresponding coupling elements, wherein one or more coupling elements of the door can be coupled to one or more coupling elements of the cover element. The coupling elements can, for example, form a bayonet lock.

In the beta container, objects such as closure elements or culture medium carriers can be provided to be transferred to the isolator. The objects can then be transferred or inserted into the isolator in the coupled state of the alpha port and the beta port. Furthermore, objects can also be transferred or inserted from the isolator into the beta container in the coupled state in order to transfer these objects out of the isolator.

When the alpha and beta ports are in the coupled state, when objects are transferred from the beta container to the isolator or from the isolator to the beta container, the objects are transferred in one transfer direction through the alpha port opening and the beta port opening, respectively. In particular, the transfer direction can be parallel to a direction extending from the inner side to the outer side of the beta port.

According to the application, an additional functional element is provided for the beta container. The functional element can be arranged on the beta container. The functional element serves to improve the transfer of objects between the beta container and the isolator. In particular, the functional element serves to improve the transfer from the beta container to the isolator and/or the transfer of objects from the isolator to the beta container.

In particular, the functional element is arranged on the beta container in a defined arrangement. In the defined arrangement, the functional element is at least partially arranged within the beta port opening. Furthermore, the bag of the beta container is arranged in the defined arrangement between the beta port and the functional element. In other words, the functional element is designed such that it can be arranged at least partially in the beta port opening and the bag then lies between the functional element and the beta port. In particular, in the defined arrangement, at least a part of the functional element extends into the beta port opening. In the defined arrangement, the functional element is arranged on the outside of the bag. The bag thus separates the functional element from the internal volume of the beta container.

The functional element has the functional element opening. With regard to the functional element opening, a radial direction, an axial direction and a tangential direction, also called the circumferential direction, can be defined. The radial direction is a direction that extends radially outwards from the opening, in particular a center point of the functional element opening. The axial direction is a direction in which the functional element opening extends through the functional element. The circumferential direction is a direction that extends around the functional element opening. The radial direction, the axial direction and the circumferential direction are perpendicular to each other. In the defined orientation, the axial direction extends parallel to the transfer direction.

The functional element opening is smaller than the beta port opening. In particular, a cross-sectional geometry of the functional element opening perpendicular to the axial direction is smaller than a cross-sectional geometry of the beta port opening perpendicular to a direction from the outer side to the inner side. In particular, a diameter of the functional element opening is smaller than a diameter of the beta port opening.

The functional element can have a functional element body. The functional element body can surround the functional element opening. The functional element body can extend outwardly from the functional element opening in the radial direction. The functional element opening can in particular be formed as a recess in the functional element body. The functional element can have a functional element flange surrounding the functional element opening. In particular, the functional element body can have or form the functional element flange.

The functional element can have a first axial end and a second axial end in the axial direction, which are arranged on opposite sides of the functional element. The functional element flange can be arranged at the first axial end. The functional element flange surrounds the functional element opening at the first axial end. The functional element body can be smaller in the radial direction at the first axial end than at the second radial end. In particular, an outer diameter of the functional element body at the first axial end can be smaller than an outer diameter of the functional element body at the second axial end. Preferably, at least the outer diameter of the functional element body at the first axial end is smaller than the beta port opening. This allows the functional element to be arranged at least partially in the beta port opening. In particular, in the defined arrangement, at least the first axial end of the functional element is arranged in the beta port opening. In the defined arrangement, the functional element is arranged on the beta container such that the first axial end faces the alpha port in the coupled state of the alpha port and the beta port. In particular, in the defined arrangement, the functional element opening is arranged within the beta port opening. In other words, the part of the functional element that extends into the beta port opening has the functional element opening.

In order to bring the functional element into the defined arrangement, the functional element is first arranged so that the bag extends through the functional element opening. The functional element will then be inserted into the beta port opening from the side of the beta port main body facing the internal volume. However, since the functional element is arranged outside the bag, the bag is partially brought or folded into the beta port opening. In particular, the part of the functional element that extends into the beta port opening is enclosed by the bag wall.

The bag of the beta container is then arranged in the defined arrangement in the beta port opening such that the bag extends through the functional element opening, is folded around the functional element flange and then extends back out of the beta port opening.

Objects such as closure elements can be provided in the beta container, particularly in its internal volume. When the beta port is coupled to the alpha port, these objects can then be inserted into the isolator. To do this, they must be transferred through the beta port opening and the alpha port opening.

When transferring objects in a transfer system between a beta container and an isolator, it is known, among other things, that contact of the objects to be transferred with a so-called “ring of concern” should be avoided. The “ring of concern” includes, for example, the contact surfaces between the alpha port and the beta port, in particular a circumferential line of the seal between the alpha port and the beta port. It is particularly desirable to avoid contact of the objects with the beta port opening and the alpha port opening as far as possible or completely.

For example, the prior art when using sterile bags as beta containers has previously involved the use of an internal tube that can be removed from the sterile bag when the alpha and beta ports are in the coupled state in order to bridge the “ring of concern.” However, the tube must be removed from the sterile bag using a gloved hand from within the isolator. This means that additional manual operation is necessary, which can lead to the potential spread of germs through the glove.

Using the new functional element, the “ring of concern” can be bridged in a simple and safe way. According to the application, the functional element is arranged such that at least a part of the functional element, which has the functional element opening, extends into the beta port opening, wherein a part of the bag also extends into the beta port opening and separates the functional element from the internal volume of the beta container.

On the transfer path, the objects must then pass from the internal volume through the functional element opening. In this way, a passage is created in the beta port for the objects to be transferred, through which the objects to be transferred can be brought into the isolator without contact with the beta port.

In a beta container without a functional element, an outlet cross section of these objects corresponds to the size of the beta port, in particular the cross-sectional geometry of the beta port. An outlet cross-section is the cross-sectional area available to the objects when passing through the beta port opening perpendicular to a transfer direction. The functional element opening serves to reduce this outlet cross section. In other words, this means that in a beta container with a functional element, the outlet cross section is reduced. In particular, this distances the cross-sectional area of the outlet cross section from an edge of the beta port opening. This allows contact of the objects with the beta port opening and the alpha port opening, in particular with the “ring of concern,” to be largely or completely avoided. In addition, the containers are also protected from contact with the beta port by the bag folded into the beta port. The new functional element thus bridges the “ring of concern” without the need for manual operation.

In a first refinement of the aspects, the functional element opening can be circular.

Preferably, the beta port opening and in particular also the alpha port opening can also be circular. In particular, the diameter of the functional element opening is smaller than the diameter of the beta port opening. The diameter of the functional element opening can be 20% to 80%, preferably 35% to 65%, in particular 50%, of the diameter of the beta port opening.

In a further refinement of the aspects, the functional element opening can be arranged concentrically to the beta port opening.

Preferably, the functional element opening and the beta port opening have the same shape, wherein the beta port opening is larger than the functional element opening. The term “concentric” is to be understood here as meaning that the functional element opening and the beta port opening are arranged symmetrically to a central axis.

In a further refinement of the aspects, the functional element can have a functional element body, wherein the functional element body surrounds the functional element opening.

As previously described, the functional element opening can be formed as a recess in the functional element body that extends through the functional element body.

In a further refinement of the aspects, the functional element body can be ring-shaped or funnel-shaped.

In a funnel shape, both sides of the funnel are open. The functional element opening is formed by the inner recess of the ring shape or funnel shape. Due to its ring or funnel shape, the functional element can be easily inserted into the beta port opening and serve as a passage for the objects from the internal volume.

In a further refinement of the aspects, the functional element body can be formed in several parts.

In particular, the functional element body can consist of a plurality of parts, in particular of two parts. These parts can be put together to form the functional element. Each part of the functional element body can preferably be adjacent to the functional element opening. In this way, the functional element can be easily arranged in the defined arrangement. In particular, the parts can be brought to the beta container in the unassembled state and only there assembled to form the functional element body at the beta port opening in such a way that the bag extends through the functional element opening. The functional element can then be moved into the beta port opening to arrange the functional element in the defined arrangement. In particular, the functional element body can be divided into a plurality of segments in the circumferential direction around the functional element opening, each segment corresponding to one of the parts of the functional element body. In particular, the functional element body can be composed of two half shells.

In a further refinement of the aspects, the functional element body can be resilient or fan-like.

In this way, the entire functional element body, in particular the functional element opening, can be widened. This also allows the functional element to be easily arranged in the defined arrangement. In particular, the functional element can be widened in order to arrange the functional element in such a way that the bag extends through the functional element opening. The functional element can then be moved into the beta port opening to arrange the functional element in the defined arrangement. For example, the functional element can be made of an elastic material. Alternatively, the functional element can also be designed as a fan. For this purpose, the functional element body can be divided into a plurality of segments in the circumferential direction around the functional element opening, wherein the segments are coupled to one another in such a way that they can be fanned out in order to widen the functional element body and in particular the functional element opening.

In a further refinement of the aspects, the functional element can be coupled to the beta port.

In particular, the functional element can be coupled to the beta port in a form-fitting or force-fitting manner in order to hold the functional element in the defined arrangement. For example, the functional element can be clamped or coupled by means of a fastening element, a buckle or a bayonet lock.

In a further refinement of the aspects, the beta port can have a cover element for covering the beta port opening, wherein the functional element is arranged between the cover element and the internal volume.

The beta port opening can be closed using the cover element. The cover element thus serves to sealingly cover or close the beta port opening.

In a further refinement of the aspects, the beta port can have a sealing element for sealing the coupling between the alpha port and the beta port.

The sealing element can preferably be arranged on the outer side of the beta port main body. In particular, the sealing element can be arranged on the beta port flange. In particular, the beta port main body can have a groove on the outer side surrounding the beta port opening into which the sealing element is inserted. The sealing element can, for example, be a lip seal. In the coupled state, the sealing element can be arranged between the alpha port flange and the beta port flange. In this way, the internal volume of the beta container and the interior of the isolator are sealed against the outer environment of the isolator.

In a further refinement of the aspects, the bag can extend through the functional element opening in the defined arrangement.

The functional element is arranged in particular outside the internal volume of the beta container. In the defined arrangement, the functional element then forms a safe passage through the beta port without the functional element coming into contact with the environment of the internal volume.

In a further refinement of the aspects, the bag can extend into the beta port opening in the defined arrangement.

In this way, contact with the beta port can be avoided.

In a further refinement of the aspects, the bag can have a bag wall, wherein the bag wall in the defined arrangement encloses the functional element in the beta port opening.

As previously explained, the bag wall can be made of a resilient or flexible material, in particular a plastics material. The bag wall separates the functional element from the internal volume. In particular, the bag wall extends through the functional element opening and is then folded back around the functional element body.

In a further refinement of the aspects, the isolator can have an interior and an isolator wall enclosing the interior, wherein the alpha port is arranged on the isolator wall, wherein the beta port is couplable to the alpha port from an outer environment of the isolator.

When the alpha port and the beta port are coupled together, objects can be transferred between the beta container and the isolator.

In a further refinement of the aspects, the alpha port can be arranged on a vertical wall surface of the isolator wall.

The vertical wall surface in the isolator extends parallel to a vertical direction. As previously described, the alpha port can be arranged on a vertical wall due to the functional element. The alpha port therefore does not have to be arranged on a bay window.

In a further refinement of the aspects, the barrier system can further comprise a feed device in the interior of the isolator, wherein the feed device can be arranged from the inside on the alpha port.

The feed device is used to feed objects, in particular closure elements, into the isolator. If the feed device is arranged at the alpha port, the objects from the beta container can be fed into the isolator through the alpha and beta ports via the feed device. The feed device can have a tube or a chute. The feed device can comprise an arm which is pivotably arranged on the isolator wall, preferably above the alpha port.

In a further refinement of the aspects, the feed device can comprise a tube, wherein a first, open end of the tube can be arranged at the alpha port, in particular wherein the tube is cylindrical.

In particular, the first end of the tube can extend into the alpha port opening. The objects from the beta container can then be inserted into the isolator via the tube. The tube has a first open end and a second open end. During transfer, the objects enter the tube at the first, open end and then come out again of the tube again at the second, open end.

In a further refinement of the aspects, the tube of the feed device can be arranged on the alpha port such that, in the coupled state, it extends through an alpha port opening of the alpha port to the beta port opening, in particular wherein the first end of the tube can be arranged on a functional element flange of the functional element.

As a result, objects that pass through the functional element opening from the internal volume of the beta container go directly into the tube of the feed device. This ensures that the objects do not come into contact with the ring of concern.

In a further refinement of the aspects, the tube of the feed device can be arranged on the alpha port in such a way that the tube extends obliquely to a horizontal direction, in particular wherein the first end of the tube has an elliptical shape.

In particular, the second end of the tube is arranged lower in the vertical direction than the first end of the tube. This allows objects to be fed through the tube by means of gravity. If the alpha port is arranged on a vertical wall surface, the alpha port opening is oriented horizontally. In other words, the transfer direction in this case is parallel to a horizontal direction. In order to ensure that the first end of the tube can still be arranged flat on the functional element flange, the first end of the tube can be cut off correspondingly obliquely. This gives the first end of the tube an elliptical shape. In particular, the diameter of the functional element opening is equal to or smaller than the smallest diameter of the elliptical shape of the first end of the tube.

It is understood that the features mentioned above and those to be explained below can be used not only in the combination specified in each case, but also in other combinations or on their own without departing from the scope of the present invention.

### DRAWING

Exemplary embodiments of the application are shown in the drawings and are described in more detail in the following description. In the drawings:

FIG. 1 is an isometric view of a first embodiment of an isolator system;

FIG. 2 is a longitudinal sectional view of the isolator system of FIG. 1;

FIG. 3 is an isometric view of the isolator system from FIG. 1 with feed device pivoted away;

FIG. 4 is an isometric view of a transfer system of the isolator system of FIG. 1;

FIG. 5 is an isometric view of a rear side of the transfer system of FIG. 4;

FIG. 6 is a longitudinal sectional view of the transfer system of FIG. 4;

FIG. 7 is an isometric view of an alpha port of the transfer system of FIG. 4;

FIG. 8 is an isometric view of a rear side of the alpha port of FIG. 7;

FIG. 9 is an isometric view of a beta container of the transfer system of FIG. 4;

FIG. 10 is a longitudinal sectional view of the beta container of FIG. 9 without functional element;

FIG. 11 is an isometric view of the beta container from FIG. 9 with functional element;

FIG. 12 is an isometric view of a functional element of the beta container of FIG. 9; and

FIG. 13 is an isometric view of a rear side of the functional element of FIG. 12.

### DETAILED DESCRIPTION

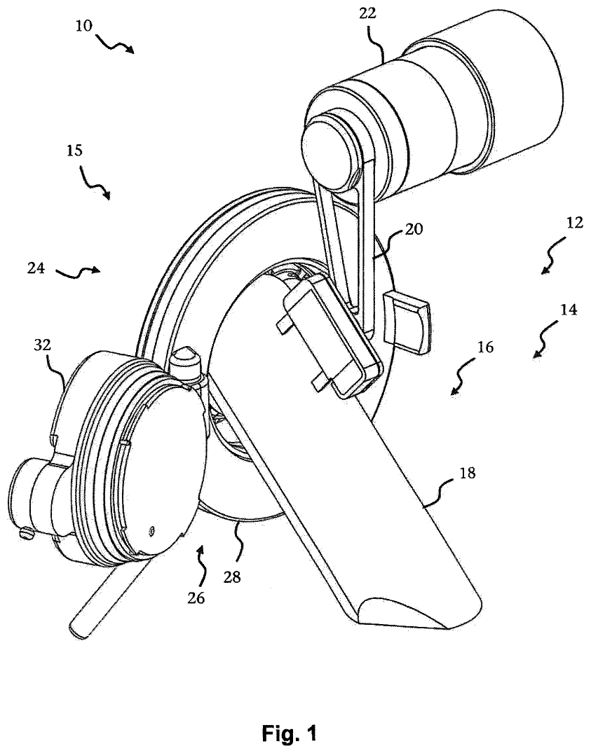

FIGS. 1 to 3 show a first embodiment of an isolator system as a barrier system, designated in its entirety by the reference numeral 10.

The isolator system 10 has an isolator 12. The isolator 12 can have an interior 14. The isolator can have an isolator wall (not shown) that encloses or surrounds the interior 14. The isolator wall separates the interior 14 from an outer environment 15 surrounding the isolator 12. The isolator 12 is preferably an aseptic isolator.

The isolator system 10 can further comprise a feed device 16. The feed device 16 serves to feed objects into the interior of the isolator 12. The feed device 16 is arranged in the interior 14 of the isolator 12. The feed device 16 has a tube 18. The feed device 16 has an arm 20. A first end of the arm 20 is fixedly connected to the tube 18. A second end of the arm 22 is pivotably mounted on the isolator wall about a horizontal axis. The feed device 16 has a drive device 22. The drive device 22 is designed to pivot the arm 20 together with the tube 18 about the horizontal axis. The tube 18 has a first open end 82 and a second open end 84. The tube 18 extends from the first end 82 to the second end 84. The tube 18 is substantially cylindrical. The tube 18 is cut off obliquely at least at the first end 82. The tube 18 is thus elliptical at the first end 82. The tube 18 can also be cut off obliquely at the two ends 82, 84. The tube 18 is then elliptical at both ends 82, 84.

The isolator system 10 further comprises a transfer system 24. The transfer system 24 is used to transfer objects into the isolator or to transfer objects out of the isolator. In particular, objects such as closure elements can be inserted into the isolator via the transfer system 24.

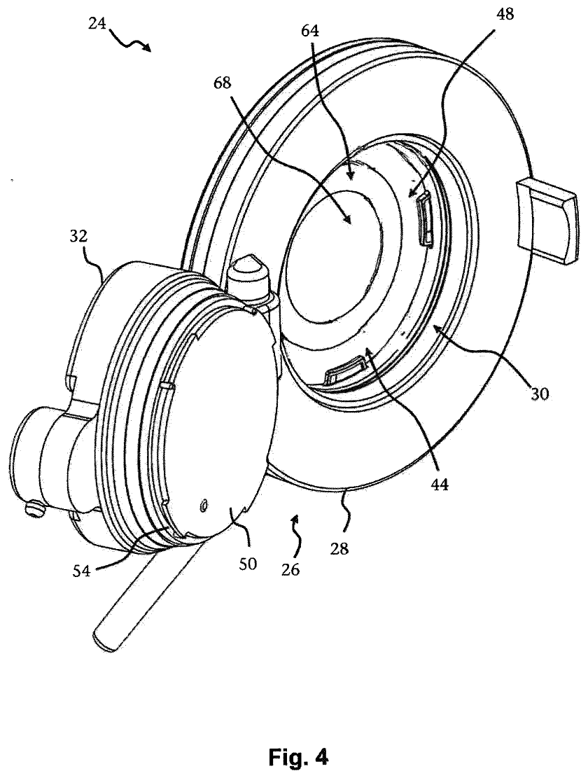

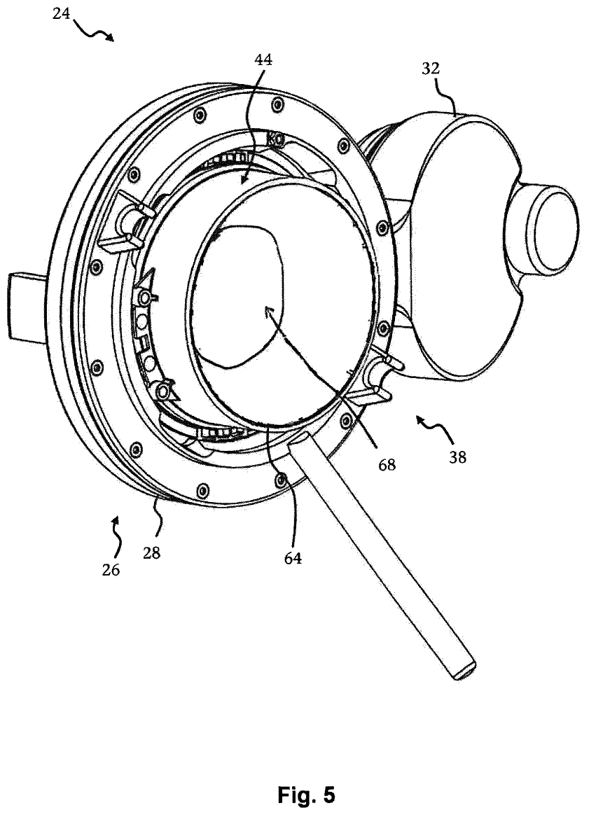

The transfer system 24 is shown in detail in FIGS. 4 to 6. The transfer system has an alpha port 26 and a beta container system 37. The beta container system 37 has a beta container 38. The alpha port 26 can be arranged on the isolator wall of the isolator 12. In particular, the alpha port 26 is arranged on a vertical wall surface of the isolator wall. The alpha port 26 forms in particular a closable passage through the isolator wall from the interior 14 to the outer environment 15.

The feed device 16 can be arranged from the inside on the alpha port 26. When the feed device 16 is arranged at the alpha port 26, the objects can be fed from the beta container 38 into the isolator 12 via the feed device 16.

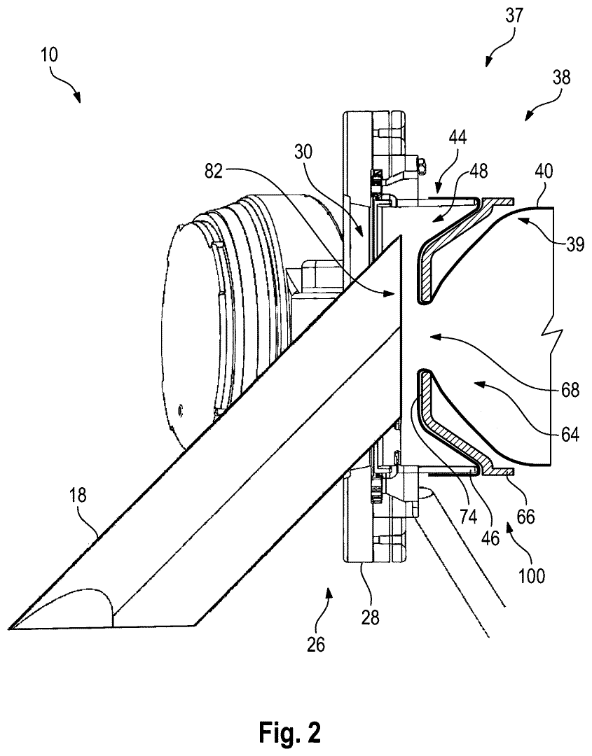

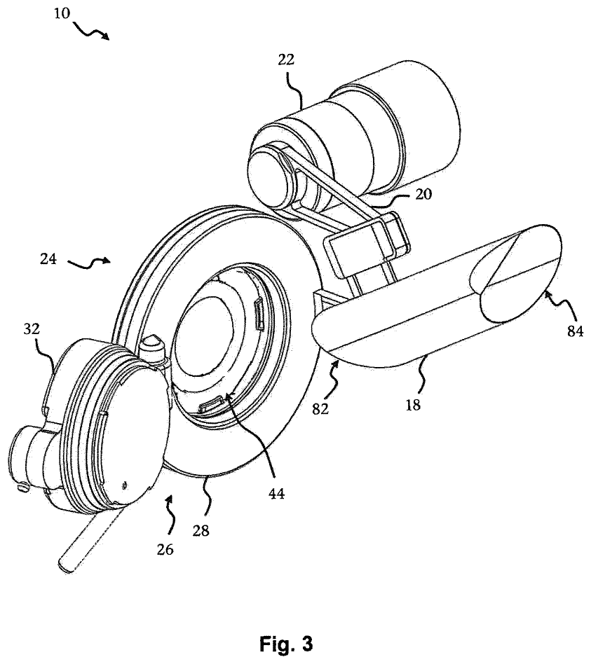

The feed device 16 is preferably arranged to be pivotable in a vertical direction above the alpha port 26. The tube 18 can be pivoted between a first position and a second position via the arm 20. In FIGS. 1 and 2, the tube 18 is arranged in the first position. In the first position, the first end 82 of the tube 18 is arranged at or in the alpha port 26. When the tube 18 is arranged in the first position, objects from the beta container 38 can be inserted into the tube 18. In FIG. 3, the tube 18 is arranged in the second position. In the second position, the first end 82 of the tube 18 is not arranged at the alpha port 26, but is moved away from it or spaced apart from the alpha port 26. When the tube 18 is arranged in the second position, the door 32 can be opened and closed.

In the first position of the tube 18, the first end 82 of the tube 18 is arranged higher in the vertical direction than the second end 84. The tube 18 therefore extends obliquely to a horizontal direction.

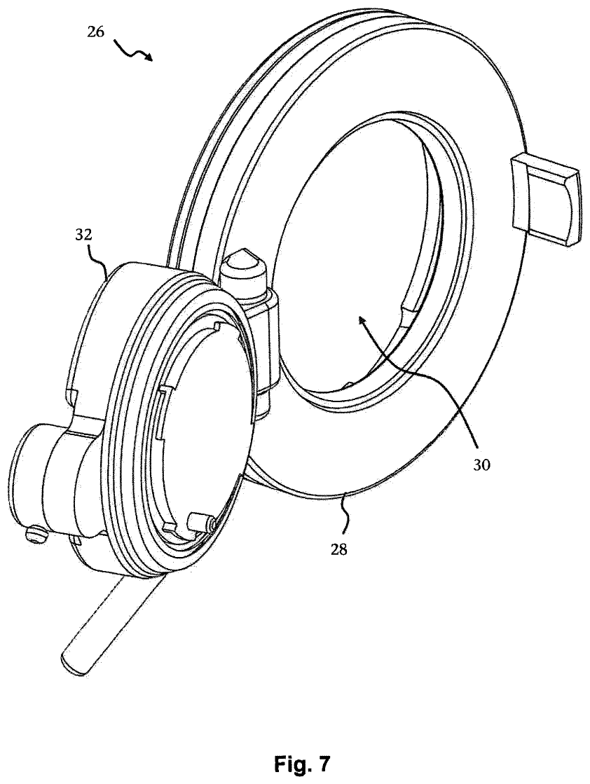

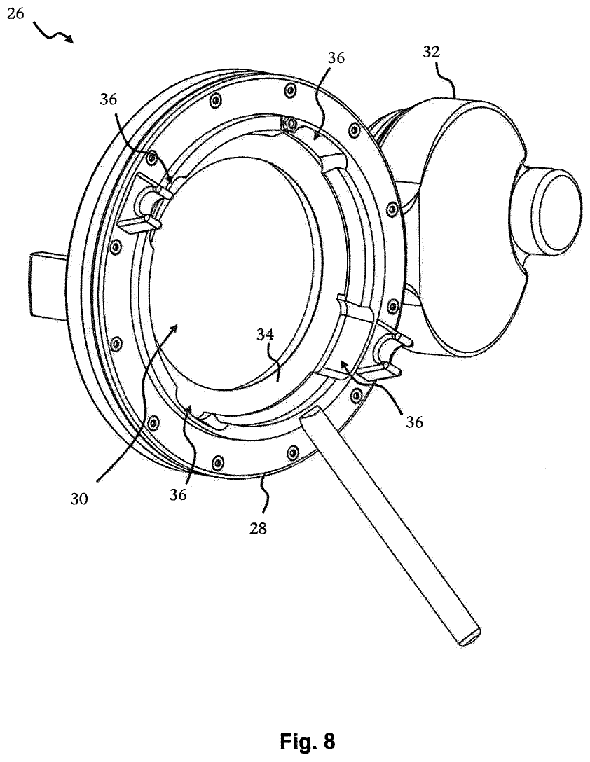

The alpha port 26 is shown in detail in FIGS. 7 and 8.

The alpha port 26 has an alpha port main body 28. The alpha port main body 28 can be ring-shaped. In particular, the alpha port main body 28 can extend through the isolator wall. The alpha port main body 28 has an inner side and an outer side. The inner side faces the interior. The outer side faces the outer environment 15. In the coupled state, the outer side is particularly facing the beta container 38

The alpha port 26 has an alpha port opening 30. The alpha port opening 30 forms a passage through the isolator wall. The alpha port opening 30 is circular. The alpha port opening 30 can be formed by a recess in the alpha port main body 28 that extends from the inner side to the outer side of the alpha port main body 28. The alpha port main body 28 surrounds the alpha port opening 30.

The alpha port 26 also has a door 32. The door 32 is arranged at the alpha port opening 30. The door 32 is used to open and close the alpha port opening 30. The door 32 is arranged movably, in particular pivotably, on the alpha port main body 28. The door 32 can be moved to open or close the alpha port opening 30. In a closed state, the door 32 tightly closes the alpha port opening 30. For example, the alpha port 26 can have a drive device (not shown) to move the door.

The alpha port 26 has an alpha port flange 34. The alpha port flange 34 is arranged on the outer side of the alpha port main body 28. In particular, the alpha port main body 28 can form the alpha port flange 34 on the outer side. The alpha port flange 34 surrounds the alpha port opening 30 on the outer side.

The alpha port 26 can further comprise one or more coupling elements 36. The coupling elements 36 serve to couple the alpha port 26 to the beta container 38. The coupling elements 36 are arranged on the alpha port main body 28, in particular on the alpha port flange 34.

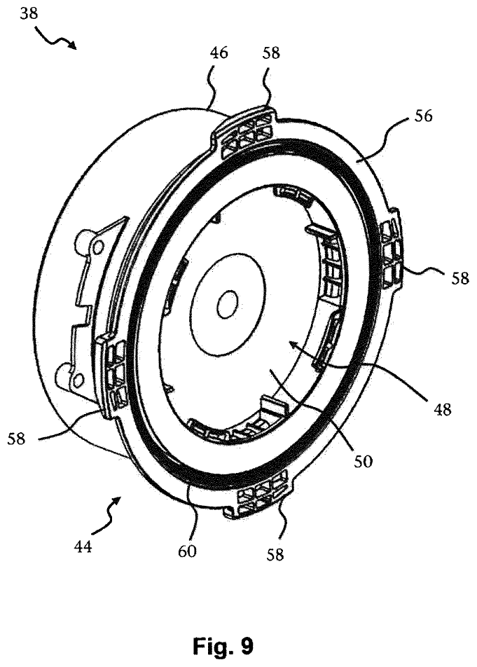

The beta container 38 is shown in detail in FIGS. 9 to 11.

The beta container 38 has a bag 39. The bag has a bag wall 40. The bag 39 can, for example, be designed to be flexible. In particular, the bag 39 can be a flexible bag. The bag 39 can be made of plastics material. The bag 39 can, for example, be a sterile bag.

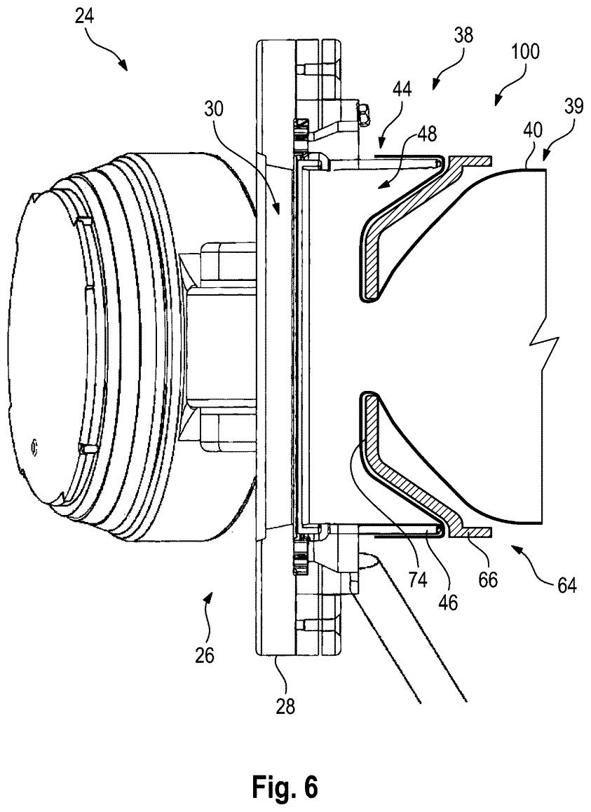

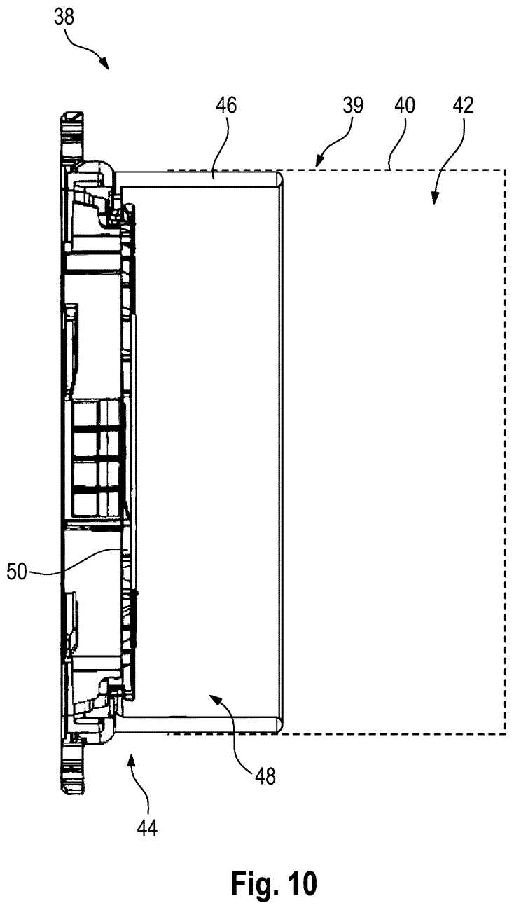

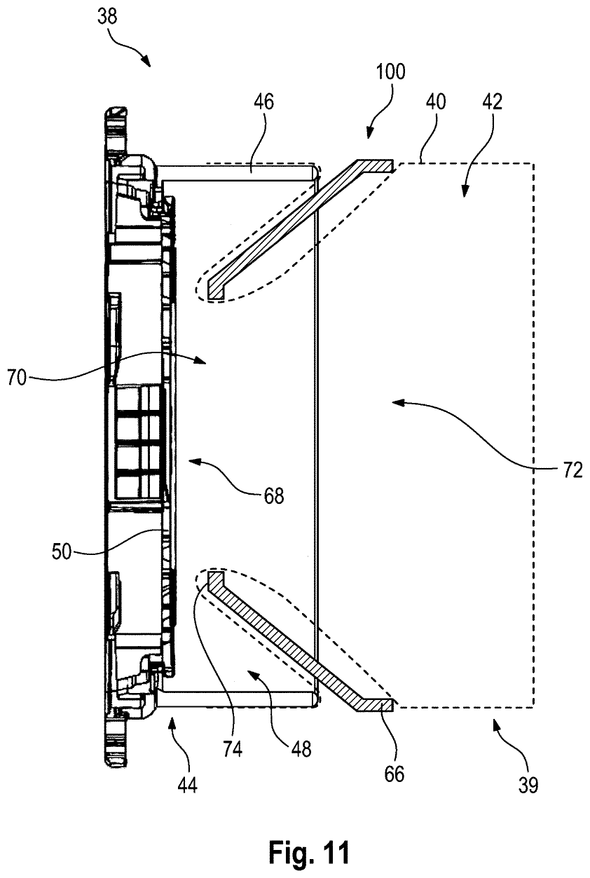

The beta container 38 further has an internal volume 42. For example, objects such as closure elements can be arranged in the internal volume 42. The bag 39 surrounds or encloses the internal volume 42. In particular, the bag 39 isolates the internal volume 42 from the outer environment 15 surrounding the beta container 38. The bag 39 and the internal volume 42 are schematically sketched in FIGS. 10 and 11 and in FIGS. 2 and 6. In the remaining figures of the isolator system 10 of the first embodiment, the bag 39 and the internal volume 42 are not shown for illustrative purposes.

The beta container 38 has a beta port 44. The beta port 44 can be arranged on the bag 39 of the beta container 38. In particular, the beta port 44 can be connected to the bag 39. The alpha port 26 and beta port 44 can be coupled together. In particular, the beta port 44 can be coupled from the outer environment 15 to the alpha port 26. In a coupled state of the alpha port 26 and the beta port 44, the internal volume 42 of the beta container 38 is connected to the interior 14 of the isolator 12.

The beta port 44 has a beta port main body 46. The beta port main body 46 can preferably be ring-shaped. In particular, the beta port main body 46 can extend through the bag wall 40. The beta port main body 46 has an inner side and an outer side. The inner side faces the internal volume 42. The outer side faces the outer environment 15. In the coupled state, the outer side in particular faces the alpha port 26.

The beta port 44 has a beta port opening 48. The beta port is connected to the bag 39. The beta port opening 48 forms a passage through the bag wall 40. In other words, the internal volume 42 is accessible from the outside through the beta port opening 48. The beta port opening 48 is circular in shape. The beta port opening 48 can be formed by a recess in the beta port main body 46 that extends from the inner side to the outer side of the beta port main body 46. In particular, the beta port main body 46 surrounds the beta port opening 48. In the coupled state, the alpha port opening 30 and the beta port opening 48 are arranged concentrically to each other.

The beta port opening 48 and the alpha port opening 30 are substantially the same size. In particular, the beta port opening 48 and the alpha port opening 30 can have the same diameter.

The beta port 44 has a beta port flange 56. The beta port flange 56 is arranged on the outer side of the beta port main body 46. In particular, the beta port main body 46 can form the beta port flange 56 on the outer side. The beta port flange 56 surrounds the beta port opening 48 on the outer side. in the coupled state, the alpha port flange 34 and the beta port flange 56 are in circumferential contact with each other.

The beta port 44 can further include a sealing element 60. The sealing element 60 serves to seal the coupling between the alpha port 26 and the beta port 44. The sealing element 60 can be arranged on the outer side of the beta port main body 46. In particular, the sealing element 60 can be arranged on the beta port flange 56. In particular, the beta port flange 56 can have on the outer side a groove surrounding the beta port opening 48, into which the sealing element 60 is inserted. The sealing element can, for example, be a lip seal. In the coupled state, the sealing element 60 is arranged between the alpha port flange 34 and the beta port flange 56. In this way, the internal volume 42 of the beta container 38 and the interior 14 of the isolator 12 are sealed from the outer environment 15.

The beta port 44 has a cover element 50. The cover element 50 can be arranged on or in the beta port opening 48. The cover element 50 serves to cover or close the beta port opening 48.

When the cover element 50 is arranged on or in the beta port opening 48 and covers the beta port opening 48, the beta container 38 is closed. The beta container 38 is then in a closed state. In FIG. 9, the beta container 38 is shown in the closed state.

When the cover element 50 is not arranged on or in the beta port opening 48 and does not cover the beta port opening 48, in particular when the cover element 50 is removed from the beta port opening 48, the beta container 38 is open. The beta container 38 is then in an opened state. In FIGS. 1 to 6, the beta container 38 is shown without the cover element 50 in the opened state.

The cover element 50 can be detachably coupled to the beta port main body 46 or the beta port flange 56. When the cover element 50 is coupled to the beta port main body 46 or the beta port flange 56, the cover element 50 closes the beta port opening 48. For coupling, the cover element 50 and the beta port main body 46 can have corresponding coupling elements. In particular, the beta port main body 46 can have projections 52 in the beta port opening 48 and the cover element can have corresponding receptacles 54 for the projections 52. To secure the cover element 50 to or in the beta port opening 48, the projections 52 can be brought into engagement with the receptacles.

The beta port 44 can further comprise one or more coupling elements 58. The coupling elements 58 serve to couple the beta port 44 to the alpha port 26. In particular, the coupling elements 58 of the beta port can couple to the coupling elements 36 of the alpha port in order to couple the alpha port and the beta port together. The coupling elements 58 are arranged on the beta port main body 46, in particular on the beta port flange 56.

The door 32 and the cover element 50 can be coupled together. When the door 32 and the cover element 50 are coupled, the door 32 and the cover element 50 can be moved together. In particular, they can be moved in such a way that the alpha port opening 30 and the beta port opening 48 can be opened and closed together. In particular, the door 32 and the cover element 50 can be coupled together when the alpha port 26 and the beta port 44 are coupled together. This allows the alpha port opening 30 and the beta port opening 48 to be opened and closed together when the alpha port 26 and the beta port 44 are coupled together.

For coupling purposes, the door 32 and the cover element 50 can have corresponding coupling elements, wherein one or more coupling elements of the door 32 can be coupled to one or more coupling elements of the cover element 50.

Objects, such as closure elements, which are to be transferred into the isolator 12 can be provided in the beta container 38. The objects can then be transferred or inserted into the isolator 12 in the coupled state of the alpha port 26 and the beta port 44. In particular, the objects can be fed from the beta container through the beta port 44, the alpha port 26 and the feed device 16 into the isolator.

The beta container system 37 further comprises a functional element 64. The functional element 64 is used to guide the transfer of objects between the beta container and the isolator. For this purpose, the functional element 64 can be arranged on the beta container 38 in a defined arrangement 100.

In the defined arrangement 100, the functional element is arranged at least partially within the beta port opening 48. Furthermore, the bag 39 of the beta container 38 is arranged in the defined arrangement 100 between the beta port 44 and the functional element 64. In FIGS. 1 to 6, 9 and 11, the functional element 64 is shown in the defined arrangement 100. In FIG. 10, the beta container 38 is shown without the functional element 64.

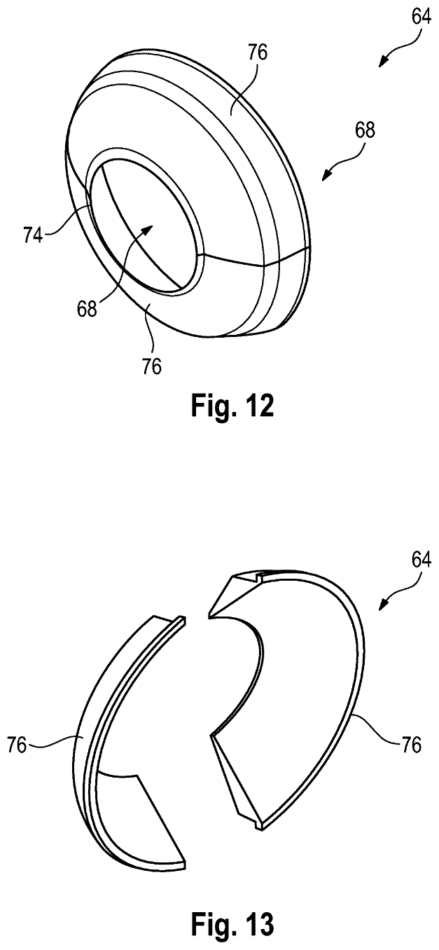

The functional element 64 is shown in detail in FIGS. 12 and 13.

The functional element 64 has a functional element body 66. The functional element body 66 is preferably ring-shaped or funnel-shaped. The functional element 64 has a functional element opening 68. The functional element opening 68 is circular. The functional element opening 68 is smaller than the beta port opening 48. In particular, a diameter of the functional element opening 68 is smaller than a diameter of the beta port opening 48. The functional element opening 68 can be arranged concentrically to the beta port opening 48. In particular, the functional element opening 68 is arranged concentrically to the beta port opening 48 in the defined arrangement.

The functional element opening 68 is formed as a recess in the functional element body 66. The functional element opening 68 extends in an axial direction through the functional element body 66. The functional element body 66 surrounds the functional element opening 68. The functional element body 66 extends radially outward from the functional element opening 68.

The functional element 64 extends in the axial direction from a first axial end 70 and a second axial end 72. When the beta port 44 is coupled to the alpha port 26, the first axial end 70 faces the alpha port 26 and the second axial end 72 faces away from the alpha port 26. In particular, an outer diameter of the functional element body 66 at the first axial end 70 is smaller than an outer diameter of the functional element body 66 at the second axial end 72.

The functional element 64 has a functional element flange 74. The functional element body has the functional element flange 74 at the first axial end 70. The functional element flange 74 surrounds the functional element opening 68 at the first axial end 70.

The functional element body 66 is formed from two parts 76. The two parts 76 can be assembled to form the functional element body 66. Each part 76 is adjacent to the functional element opening 68. In other words, the functional element body 66 is divided into two segments in the circumferential direction around the functional element opening 68, each segment corresponding to one of the parts 76. The two parts are designed in particular as two half shells. In FIG. 12, the two parts 76 are shown in the assembled state and form the functional element body 66. In FIG. 13, the two parts 76 are shown separated in the unassembled state.

In the defined arrangement 100, the bag 39 extends through the functional element opening 68. In the defined arrangement 100, the bag 39 extends in particular into the beta port opening 48. In the defined arrangement 100, the bag wall 40 encloses the part of the functional element 64 that is arranged in the beta port opening 48.

In particular, the functional element 64 can only be arranged in the defined arrangement when the beta port 44 and the alpha port 26 are coupled to one another. In FIG. 10, the beta container 38 is shown without the functional element 64. In order to bring the functional element 64 into the defined arrangement 100, the functional element 64 is first arranged such that the bag 39 extends through the functional element opening 68. For this purpose, the two parts 76 on the side of the beta port main body 46 facing the internal volume 42 are assembled around the bag 39 in such a way that they form the functional element body 66 and the bag 39 extends through the functional element opening 68. Then, the functional element 64 is moved into the beta port opening 48 from the side of the beta port main body 46 facing the internal volume 42. However, since the functional element 64 is arranged outside the bag 39, the bag 39 is partially brought into the beta port opening 48. The bag 39 is then arranged in the defined arrangement 100 of the functional element 64 in the beta port opening 48 such that the bag 39 extends through the functional element opening 68, is folded around the functional element flange 74 and then extends back out of the beta port opening 48. This is shown in FIGS. 2, 6 and 11.

As previously described, objects, such as closure elements, can be provided in the internal volume 42 of the beta container 38, wherein these objects can then be inserted or fed into the interior of the isolator 12 by means of the transfer system 24 and the feed device 16. To do this, the beta port 44 is first coupled to the alpha port 26 and the door 32 is opened together with the cover element 50. Then the tube 18 of the feed device 16 can be pivoted into the first position. This achieved state is shown in FIGS. 1 and 2. The objects can then be fed from the internal volume 42 through the beta port opening 48, the alpha port opening 30 and the tube 18 into the isolator 12. The functional element 64 is arranged such that the objects must pass through the functional element opening 68 when the objects pass through the beta port opening 48.

Since the alpha port 26 is arranged on a vertical wall surface of the isolator wall, the alpha port opening 30 extends in a horizontal direction from the inner side to the outer side of the alpha port main body 28. In the first position, the tube 18 is arranged obliquely to the horizontal direction. As a result, the tube 18 is aligned obliquely to the alpha port opening 30 in the first position.

In the first position of the tube 18, the first end of the tube 82 extends into the alpha port opening 30 to the beta port opening 48. The first end of the tube 82 extends in particular to the functional element flange 74. The first end of the tube 82 rests against the functional element flange 74. In particular, the functional element opening 68 is designed such that it is smaller than or equal to the opening of the tube 18 at the first end 82. The functional element opening 68 is aligned with the opening of the tube 18 at the first end 82. As a result, all objects leaving the functional element opening 68 at the first axial end 70 pass directly into the tube 18.

It is to be understood that the foregoing is a description of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “e.g.,” “for instance, “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

1. A functional element for a beta container system, comprising a functional element opening, wherein the functional element opening is smaller than a beta port opening of a beta port of a beta container of the beta container system; the functional element can be arranged on the beta container in a defined arrangement; the functional element is arranged at least partially in the beta port opening in the defined arrangement; and a bag of the beta container is arranged in the defined arrangement between the beta port and the functional element.

2. The functional element according to claim 1, wherein the functional element opening is circular.

3. The functional element according to claim 1, wherein the functional element opening can be arranged concentrically to the beta port opening.

4. The functional element according to claim 1, wherein the functional element has a functional element body that surrounds the functional element opening.

5. The functional element according to claim 4, wherein the functional element body is ring-shaped or funnel-shaped.

6. The functional element according to claim 4, wherein the functional element body is formed in several parts.

7. The functional element according to claim 4, wherein the functional element body is elastic or fan-like.

8. The functional element according to claim 4, wherein the functional element can be coupled to the beta port.

9. A beta container system for a transfer system, comprising a beta container and a functional element, wherein the functional element has a functional element opening; the functional element opening is smaller than a beta port opening of a beta port of the beta container of the beta container system; the functional element can be arranged on the beta container in a defined arrangement; the functional element is arranged at least partially in the beta port opening in the defined arrangement; a bag of the beta container is arranged in the defined arrangement between the beta port and the functional element; the beta container has an internal volume, the bag which encloses the internal volume, and the beta port with the beta port opening; and the beta port can be coupled to an alpha port of the transfer system.

10. The beta container system according to claim 9, wherein the beta port includes a cover element for covering the beta port opening.

11. The beta container system according to claim 9, wherein the beta port has a sealing element for sealing a coupling between the alpha port and the beta port.

12. The beta container system according to claim 9, wherein the bag in the defined arrangement extends through the functional element opening.

13. The beta container system according to claim 9, wherein the bag in the defined arrangement extends into the beta port opening.

14. The beta container system according to claim 9, wherein the bag has a bag wall, the bag wall in the defined arrangement encloses the functional element in the beta port opening.

15. A transfer system, comprising an alpha port and a beta container system, wherein the beta container system comprises a beta container and a functional element; the functional element has a functional element opening; the functional element opening is smaller than a beta port opening of a beta port of the beta container of the beta container system; the functional element can be arranged on the beta container in a defined arrangement; the functional element is arranged at least partially in the beta port opening in the defined arrangement; a bag of the beta container is arranged in the defined arrangement between the beta port and the functional element; the beta container has an internal volume, the bag which encloses the internal volume, and the beta port with the beta port opening; and the beta port can be coupled to an alpha port of the transfer system.

16. A barrier system, comprising a transfer system, wherein the transfer system comprises an alpha port and a beta container system; the beta container system comprises a beta container and a functional element; the functional element has a functional element opening; the functional element opening is smaller than a beta port opening of a beta port of the beta container of the beta container system; the functional element can be arranged on the beta container in a defined arrangement; the functional element is arranged at least partially in the beta port opening in the defined arrangement; a bag of the beta container is arranged in the defined arrangement between the beta port and the functional element; the beta container has an internal volume, the bag which encloses the internal volume, and the beta port with the beta port opening; and the beta port can be coupled to the alpha port of the transfer system.

17. The barrier system according to claim 16, wherein the barrier system further comprises an isolator, the isolator has an interior and an isolator wall enclosing the interior, the alpha port is arranged on the isolator wall, and the beta port is couplable to the alpha port from an outer environment of the isolator.

18. The barrier system according to claim 17, wherein the alpha port is arranged on a vertical wall surface of the isolator wall.

19. The barrier system according to claim 17, wherein the barrier system further comprises a feed device in the interior of the isolator, the feed device can be arranged from the inside on the alpha port.

20. The barrier system according to claim 19, wherein the feed device comprises a tube, a first open end of the tube can be arranged at the alpha port.

21. The barrier system according to claim 20, wherein the tube is cylindrical.

22. The barrier system according to claim 20, wherein the tube of the feed device can be arranged on the alpha port such that, in the coupled state, the tube extends through an alpha port opening of the alpha port to the beta port opening.

23. The barrier system according to claim 22, wherein the first open end of the tube can be arranged on a functional element flange of the functional element.

24. The barrier system according to claim 20, wherein the tube of the feed device can be arranged on the alpha port such that the tube extends obliquely to a horizontal direction.

25. The barrier system according to claim 24, wherein the first open end of the tube has an elliptical shape.

26. The barrier system according to claim 16, wherein the barrier system is an isolator system.
